SEQUENCE LISTING (8 sequences)

SEQ ID NO: 1 - DNA, 34 bp, Artificial Sequence
  DENV forward primer
aacagcatattgacgctgggagagaccagagatc

SEQ ID NO: 2 - DNA, 36 bp, Artificial Sequence
  DENV reverse primer
attcaacagcaccattccattttctggcgttctgtg

SEQ ID NO: 3 - DNA, 34 bp, Artificial Sequence
  SARS-COV2 forward primer
cttcaacctaggacttttctattaaaatataatg

SEQ ID NO: 4 - DNA, 33 bp, Artificial Sequence
  SARS-COV2 reverse primer
gttggttggactctaaagttagaagtttgatag

SEQ ID NO: 5 - DNA, 15 bp, Artificial Sequence
  DENV probe
tggaatgatgctgta

SEQ ID NO: 6 - DNA, 15 bp, Artificial Sequence
  SARS-COV2 probe
agtctacagcatctg

SEQ ID NO: 7 - DNA, 24 bp, Artificial Sequence
  SARS-COV2 probe
cacagtctacagcatctgtaatgg

SEQ ID NO: 8 - DNA, 15 bp, Artificial Sequence
  Zirka virus forward primer
agcaaggggaatttg

(19) United States
(12) Patent Application Publication
(10) Pub. No.: US 2024/0182990 A1
(43) Pub. Date: Jun. 6, 2024

(54) DETECTION OF INFECTIOUS AGENT BASED ON RECOMBINASE POLYMERASE AMPLIFICATION COMBINED WITH A MAGNETIC FIELD-ENHANCED AGGLUTINATION

(71) Applicants: Etablissement Français du Sang, LA PLAINE SAINT DENIS (FR); Université de Montpellier, MONTPELLIER (FR); INSTITUT NATIONAL DE LA SANTE ET DE LA RECHERCHE MEDICALE (INSERM), PARIS (FR); HORIBA ABX SAS, MONTPELLIER CEDEX 04 (FR)

(72) Inventors: Chantal FOURNIER-WIRTH, LA PLAINE SAINT DENIS CEDEX (FR); Fanny LEON, Lézignan-la-Cèbe (FR); Jean-François CANTALOUBE, SAINT GELY DU FESC (FR); Jean-Pierre MOLÈS, COURNONSEC (FR); Elena PINCHON, LE CRES (FR); Aurélien DAYNÈS, MONTPELLIER (FR); Charly MAYRAN, Saint-Mathieu de Tréviers (FR)

(21) Appl. No.: 18/549,058

(22) PCT Filed: Mar. 9, 2021
(86) PCT No.: PCT/IB2021/000152

§ 371 Date: Sep. 5, 2023

(51) Int. Cl. C12Q 1/70; C12Q 1/6804; C12Q 1/6844; C12Q 1/689

(52) U.S. Cl. CPC C12Q 1/701; C12Q 1/6804; C12Q 1/689; C12Q 1/6844

(57) ABSTRACT

The present invention concerns a method for the molecular detection of an infectious agent based on isothermal amplification by recombinase polymerase amplification (RPA) combined with a Magnetic Field-Enhanced Agglutination (MFEA) readout.

### CROSS-REFERENCE TO RELATED APPLICATIONS

This application claims benefit under 35 USC § 371 of PCT Application No. PCT/IB2021/000152 entitled DETECTION OF INFECTIOUS AGENT BASED ON RECOMBINASE POLYMERASE AMPLIFICATION COMBINED WITH A MAGNETIC FIELD-ENHANCED AGGLUTINATION, filed on Mar. 9, 2021 by inventors Chantal Fournier-Wirth, Fanny Leon, Jean-François Cantaloube, Jean-Pierre Moles, Elena Pinchon, Aurélien Daynes and Charly Mayran,

### FIELD OF THE INVENTION

The present invention concerns a method for the molecular detection of an infectious agent based on isothermal amplification by recombinase polymerase amplification (RPA) combined with a Magnetic Field-Enhanced Agglutination (MFEA) readout.

### BACKGROUND OF THE INVENTION

Nucleic Acid Testing is commonly used for many diagnostic assays in various fields including genetic diseases, cancer or infectiology. This approach requires several sequential steps: nucleic acid extraction, amplification and detection of molecular targets. The last two steps are usually performed with sophisticated thermal cyclers with fluorescence detection by skilled personnel and in a dedicated environment for molecular biology, which is not compatible with point-of-care testing. However, various approaches are currently being tested to simplify the amplification step, or the detection step.

The inventors have designed a fast and easy-to-use DNA amplification and detection method, and demonstrated that a magnetic field-enhanced agglutination assay is compatible with, and can be combined to recombinase polymerase amplification.

Recombinase polymerase amplification (RPA) was first described in Piepenburg et al., PLoS Biol. 2006 July;4(7):e204. This technique couples isothermal recombinase-driven primer targeting of template material with strand-displacement DNA synthesis and achieves exponential amplification with no need for pretreatment of sample DNA. Magnetic field-enhanced agglutination (MFEA) consists in applying a magnetic field generated by an electromagnet to a reaction medium to accelerate the capture of a target between magnetic nanoparticles (MNPs) by a fast chaining process. The principle of MFEA was described in the international patent application WO 03/044532. The result of this agglutination performed in a homogeneous phase can then be assayed by a simple turbidimetry readout in less than 5 min.

The method, when applied to RNA viruses, enables the rapid detection within 1 hour, without the need for sophisticated laboratory automates.

### SUMMARY OF THE DESCRIPTION

The invention relates to an in vitro method for detecting an infectious agent, which comprises submitting a nucleic acid extract of a sample likely to contain the infectious agent to a recombinase polymerase amplification, followed by magnetic-field enhanced agglutination assay and determining if the infectious agent is present in the sample based on the result of the magnetic-field enhanced agglutination assay.

According to an embodiment, the method comprises:
a) Providing a nucleic acid extract of the sample likely to contain the infectious agent;
b) If the infectious agent's genomic nucleic acid is RNA, submitting the nucleic acid extract to reverse transcription, to reverse transcribe infectious agent's RNA into DNA;
c) Submitting the nucleic acid extract to recombinase polymerase amplification, using a pair of primers targeting a region of the infectious agent's DNA, wherein one primer of the primer pair is bound to a first member of a binding pair;
d) Optionally, denaturing double stranded DNAs obtained after recombinase polymerase amplification to obtain single stranded DNAs, wherein a part of the single stranded DNAs is bound to the first member of the binding pair;
e) Contacting the single stranded DNAs bound to the first member of the binding pair with (i) a first set of magnetic beads coated with a nucleic acid probe having complementarity with the single stranded DNAs bound to the first member of the binding pair, and (ii) a second set of magnetic beads coated with the second member of the binding pair;
f) Submitting the single stranded DNAs bound to the first member of the binding pair, and first and second sets of magnetic beads to magnetic-field enhanced agglutination;
g) Comparing variation of agglutination state measured before and after magnetic-field enhanced agglutination with a control to determine if the sample is positive for the infectious agent.

According to an embodiment of the method, the nucleic acid probe having complementarity with the single stranded DNAs bound to the first member of the binding pair is 5′-polythiolated and is covalently grafted to the first set of magnetic beads.

According to an embodiment of the method, the second set of magnetic beads are covered partly or totally with the second member of the binding pair.

According to an embodiment, the magnetic beads of the first and second sets of magnetic beads are magnetic micro- or nano-particles.

According to an embodiment of the method, the magnetic-field enhanced agglutination comprises 1-10 cycles of magnetization and relaxation. In an embodiment, magnetization comprises applying a magnetic field of 3-100 mT, for a duration of 1 to 300 s, and relaxation lasts 1 to 300 s. Preferably, magnetic-field enhanced agglutination comprises 2-4 cycles of magnetization at 13-17 mT, for 50-70 s, and relaxation for 20-40 s.

According to an embodiment of the method, the first member of the binding pair is biotin, and the second member of the binding pair is avidin, or an avidin derivative, or an anti-biotin antibody.

The invention also relates to an in vitro method for determining if a subject is infected with an infectious agent, which comprises implementing a method for detecting an infectious agent as defined herein on a biological sample of the subject likely to contain nucleic acids of the infectious agent; and determining that the subject is or has been infected if the infectious agent is present in the biological sample.

The invention further provides for an in vitro method for determining if a subject is or has been infected with an infectious agent, which comprises:
a. implementing a method for detecting the infectious agent as defined herein on a nucleic acid extract of a biological sample of the subject likely to contain the infectious agent;
b. Implementing an immune assay comprising a serological assay to determine if the subject has antibodies directed against the infectious agent and/or antigens of the infectious agent, and/or a cellular assay on biological sample to determine if a cell is activated upon infection; and
c. determining that the subject is or has been infected based on the result of the method for detecting the infectious agent and or the immune assay

### BRIEF DESCRIPTION OF THE DRAWINGS

The invention will be further illustrated by the following figures and examples.

FIG. 1. Method for the rapid molecular detection of RNA viruses based on RT-RPA amplification combined with a Magnetic Field-Enhanced Agglutination readout.

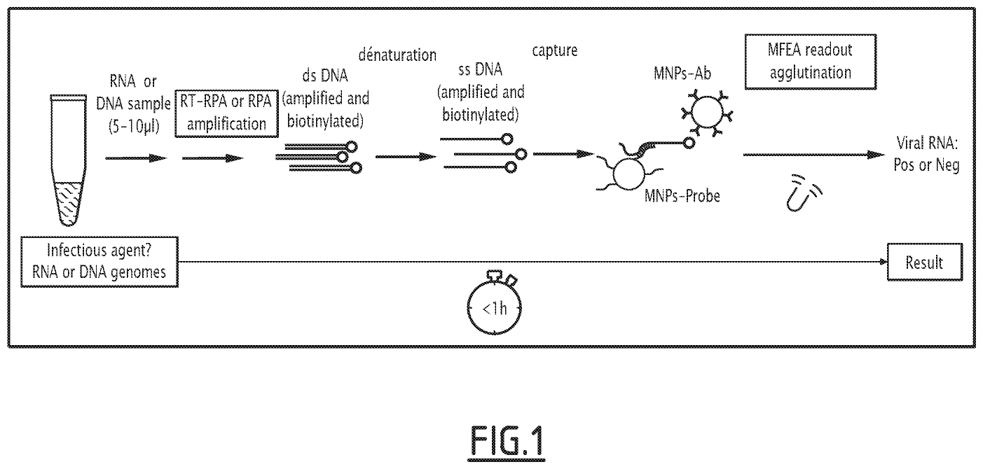

FIG. 2. Detection of DENV genomes amplified by RT-RPA. Signals were analysed for serial dilutions from 10⁶ to 1 TCID₅₀/mL of supernatants from cell cultures infected with DENV. Human plasma samples from blood donors were used as negative plasma samples (neg). After extraction and amplification using a RT-RPA, DENV genomes were analysed using a MFEA readout.

FIG. 3. Molecular MFEA readout for DENV RNA(−) and DENV RNA(+) plasma samples. Negative plasma from donors (n=30) and positive plasma samples from patients (n=31) were assayed. The turbidity signal is expressed as the difference of optical density at 650 nm (D OD₆₅₀ₙₘ) measured before and after the three magnetization cycles. The limit of detection (LOD) s taken as the mean value of blank samples plus three standard deviations. Individual points of the scatterplot represent the ratio of turbidity signal/LOD calculated for one sample by the molecular MFEA readout. Data are expressed as median ratios with interquartile ranges. **** p value <0,0001; unpaired t test

FIG. 4. Detection of DENV genomes amplified by RT-RPA. Signals were analysed for serial dilutions from 100 to 1 TCID₅₀/mL of supernatants from cell cultures infected with DENV. Human plasma samples from blood donors were used as negative plasma samples (neg), and 1000 pM of a synthetic single-stranded DNA, fully complementary to the nucleic acid probe bound to MNPs, as a positive control. After extraction and amplification using a RT-RPA, DENV genomes were analysed using a MFEA readout. (A) RT-RPA-MFEA method with thermal denaturation. (B) RT-RPA-MFEA method with chemical denaturation.

FIG. 5. Detection of SARS-COV2 genomes amplified by RT-RPA. Signals were analysed for serial dilutions of SARS-COV2 RNA materiel from Ct24 to Ct36, as determined by real-time PCR, and 1000 pM of a 15-mer or 24-mer synthetic single-stranded DNA, fully complementary to the nucleic acid probe bound to MNPs, as a positive control. (A) RT-PCA-MFEA method with 15-mer tetrathiolated SARS-COV2 probe. (B) RT-RPA-MFEA method with 24-mer tetrathiolated SARS-COV2 probe.

### EXAMPLES

We report here the development of a simple and rapid magnetic field-enhanced agglutination assay that detects RPA amplified products.

### Example 1: DENV and Sars-CoV2 Viruses RT-RPA Amplification Followed by MEFA

### DENV RT-RPA Amplification

The RT-RPA assay was carried out using the TwistAmp Basic kit (TwistDx, Cambridge, UK) supplemented with the SuperScript II reverse transcriptase (RT) (Thermo Fisher Scientific, Waltham, Massachusetts, USA) straight added to the mix. The assay was performed in a 50 μL reaction volume containing 5 μL of extracted RNA. Briefly, 29.5 μL of Rehydration buffer were mixed with 2.4 μL of 5′biotinylated forward primer (10 μM), 2.4 μL of reverse primer (10 μM) (see Table 1), 7.2 μL of DNase-free water and 1 μL of SuperScript II. The reaction mixture (42.5 μL) was added to a tube containing the RT-RPA enzyme mix in a lyophilized form, briefly mixed and spined. Then 5 μL of extracted RNA were added to the reaction mixture, briefly mixed and spined. Finally, the reaction was triggered by adding 2.5 μL of 280 mM magnesium acetate. After briefly mixed and spined, the tubes were placed into a thermomixer (Eppendorf, Hambourg, Allemagne) at 42° C. and incubated for 4 min, then briefly mixed and spined, and finally replaced in the thermomixer for 26 min at 42° C. After amplification, amplicons were diluted to the 10^{th} in Hybridization buffer, denatured at 95° C. during 10 min and placed in ice 2 min before incubation with MNPs-Probe.

Chemically denaturation with 0.4N NaOH at room temperature during 5 min, followed by addition of 0.4N acetic acid before incubation with MNPs-Probe, was also assayed as an alternative to thermal denaturation.

### Sars-Cov 2 RT-RPA Amplification

The 5′biotinylated forward primer and reverse primer used were as shown in Table 1. They were designed to amplify the S gene of SARS-COV2.

In order to improve the reaction, the SuperScript IV (Thermo Fisher Scientific, Waltham, Massachusetts, USA) (0.5 μL) and the RNAse H (1 μL) (New England Biolabs, Ipswich, Massachusetts, USA) were used. Furthermore, the input of extracted RNA is 10 μL to perform the Sars-Cov2 RT-RPA amplification. Amplicons diluted to the 10^{th} in Hybridization buffer were chemically denatured with 0.4N NaOH at room temperature during 5 min followed by addition of 0.4N acetic acid before incubation with MNPs-Probe.

TABLE 1
Primers for amplification of DENV or Sars-CoV2 viruses

| | | | | | |
|---|---|---|---|---|---|
| | | | Target | Amplicon | |
| Name | Function | Sequence (5′-3′) | gene | size (bp) | Reference |

Target amplification (RT-RPA)

| | | | | | |
|---|---|---|---|---|---|
| Pan- | Forward primer | 5′Biot-AAC-AGC-ATA-TTG- | 3′UTR | 97 | 1 |
| DENV | (DENV sens) | ACG-CTG-GGA-GAG-ACC- | | | |
| viruses* | | AGA-GAT-C (SEQ ID NO: 1) | | | |
| | Reverse primer | 5′ ATT-CAA-CAG-CAC-CAT- | | | |
| | (DENV rev) | TCC-ATT-TTC-TGG-CGT-TCT- | | | |
| | | GTG (SEQ ID NO: 2) | | | |
| Sars- | Forward primer | 5′Biot-CTT-CAA-CCT-AGG- | S | 161 | 2 |
| CoV2 | (Sars-Cov2 sens) | ACT-TTT-CTA-TTA-AAA-TAT- | | | |
| | | AAT-G (SEQ ID NO: 3) | | | |
| | Reverse primer | 5′ GTT-GGT-TGG-ACT-CTA- | | | |
| | (Sars-Cov2 rev) | AAG-TTA-GAA-GTT-TGA-TAG | | | |
| | | (SEQ ID NO: 4) | | | |

1. Abd El Wahed Aet al.. 2015 PloS one 10:e0129682-e0129682.
2. Xue G, et al.. 2020. Anal Chem 92:9699-9705.
*: The pair of primers pan-DENV viruses enables amplification of any DEN of serotype 1 to 4.

A non-complementary 15-mer Zika virus (ZIKV) DNA oligo-nucleotide (AGC AAG GGG AAT TTG, SEQ ID NO: 8) biotinylated at its 5′-end (Eurogentec, Angers, France) was used to control the non-specific events in the DENV and SARS-COV2 RT-RPA.

### Design of Tetrathiolated DENV and Sars-CoV2 Probes and Grafting Magnetic Nanoparticles

A generic 15-mer tetrathiolated DENV probe aimed at detecting dengue viral genomes was designed after aligning the nucleotide sequences of the NS5 gene from 53 strains of DENV. For detecting SARS-COV2, a 15-mer tetrathiolated probe (Sars-CoV2 15) and a 24-mer tetrathiolated probe (Sars-CoV2 24) were designed.

The 5′-tetrathiolated probes were synthesized on a 1 μmol-scale using a DNA synthesizer, and lyophilized before use (F. Leon et al. J. Mol. Diagn. 21 (1) (2019) 81-88; M. Lereau et al. Anal. Chem. 85 (19) (2013) 9204-9212).

The probes were separately covalently grafted on 200 nm diameter magnetic nanoparticle (MNPs) (200 nm carboxyl-adembeads, Ademtech, Pessac France). Ademtech manufactures calibrated particles (CV<20%), with high magnetic content (70% of iron oxide) and controlled surface bearing various functionalities. The 200 nm diameter nm carboxyl-adembeads, have been selected in the MFEA assay. These MNPs are monodispersed and super-paramagnetic beads composed of magnetic core encapsulated by a highly crosslinked hydrophilic polymer shell. Briefly, after washing and resuspension in Activating Buffer (AB) 1× (Ademtech, Pessac, France), 11.5 mg of MNPs were incubated for 30 min at 37° C. under agitation at 1000 rpm (ThermoMixer comfort, Eppendorf, Hamburg, Germany) with 1-ethyl-3-[3-(dimethylamino)pro-pyl] carbodiimide hydrochloride (6 mg/mL) to form an ester active intermediate. Then, the activated MNPs were incubated with amino-PolyEthyleneGlycol (PEG)-maleimide (8 mg/mL) in AB 1× for 2 h at 37° C. under agitation at 1000 rpm (ThemoMixer comfort). In parallel, 200 nmol of lyophilized polythiolated probe were incubated for 10 min at 20° C. with 100 μL of tris(2-carboxyethyl)phosphine hydrochloride (20 mM) to reduce the disulfide bonds, and 900 μL of Binding Buffer (0.1 M Na2HPO4, 0.15 M NaCl, 10 mM EDTA, pH7.2) was added. After washing with Storage Buffer (SB) 1× (Ademtech, Pessac, France), the PEG-maleimide MNPs were incubated for 3 h at 20° C. with the reduced polythiolated DENV probe (200 nmol/mL). The beads were placed on a magnet (Ademtech, Pessac, France), to remove the supernatant and were passivated by sequential incubations with 1 mL of tris HCl 1.5 M pH 8.8 for 20 min and 250 μL of a cysteine solution (80 mg/mL) for 10 min. After this blocking step, the MNPs covalently grafted with either the DENV probe, or the SARS-COV2 15 or SARS-COV2 24 probe (MNPs-Probe) were washed twice in 1 mL of SB and stored at 1% w/v in a dedicated buffer (10 mM Glycine 0.02% NaN3, 0.1% F108, pH 9) for up to 6 months at 4° C.

TABLE 2
Probes for detection of DENV or Sars-COV2 viruses by MFEA

| | | |
|---|---|---|
| DENV | Tetrathiolated | 5′ TGG-AAT-GAT-GCT-GTA (SEQ ID NO: 5) |
| | probe | |
| Sars- | Tetrathiolated | 5′ AGT-CTA-CAG-CAT-CTG (SEQ ID NO: 6) |
| CoV2 15 | probe | |
| Sars- | Tetrathiolated | 5′ CAC-AGT-CTA-CAG-CAT-CTG-TAA-TGG (SEQ ID NO: 7) |
| CoV2 24 | probe | |

### Magnetic Field-Enhanced Agglutination Assay

The device included a disposable spectrophotometric cuvette surrounded by an electromagnet that provided a 15 mT (mT) field, a LED source emitting at 650 nm and a photodiode (Daynes et al. Anal. Chem. 87 (15) (2015) 7583-7587). MNPs grafted with anti-biotin antibodies (MNPs-Ab) were prepared using a carbodiimide coupling chemistry by adding 10 μg of anti-biotin antibody (Jackson ImmunoResearch Europe LTD, Cambridge, UK) to 1 mg of MNPs. Increasing the antibody/MNPs ratio had no impact on the signal. Three cycles of magnetization (60 s) and relaxation (30 s) led to the progressive formation of aggregates. The turbidity signal was expressed as the total variation of optical density at 650 nm (Δ OD650 nm) measured before and after the three magnetization cycles.

### RESULTS

### DENV RT-RPA Amplification and MEFA

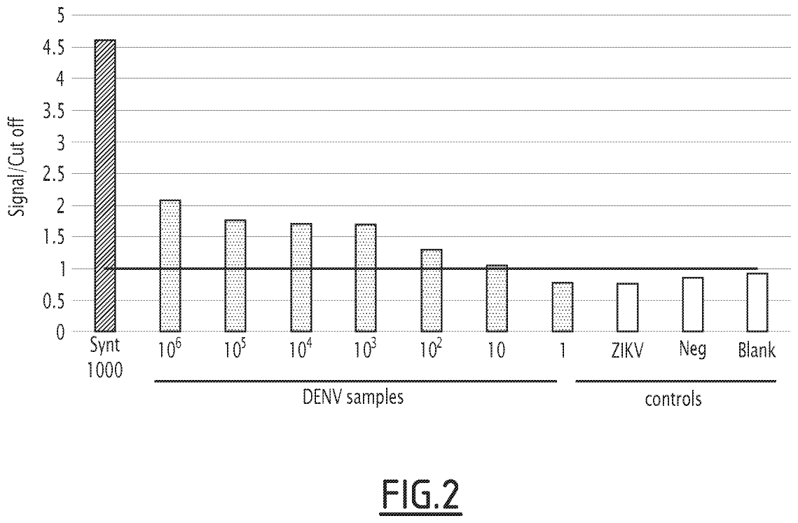

Serial dilutions from 10⁶ to 1 TCID₅₀/mL of supernatants from cell cultures infected with DENV were used to determine that the limit of sensitivity of the RT-RPA-MFEA for the dengue viruses is 10 TCID₅₀/mL (see FIG. 2).

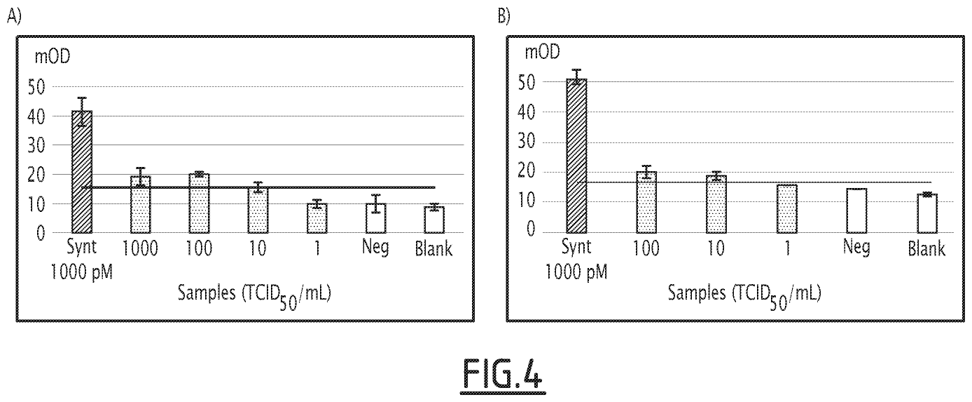

The comparison of thermal and chemical denaturation shows that thermal denaturation can be replaced by chemical denaturation without negatively impacting the sensibility of detection (FIG. 4).

A total of 31 DENV(+) clinical samples were analysed according to the method described in this example and determined as positive or negative after RT-RPA-MFEA. The set of samples included clinical samples of patients infected with dengue virus of any serotype (serotypes 1 to 4). Human plasma samples from blood donors were used as negative plasma samples.

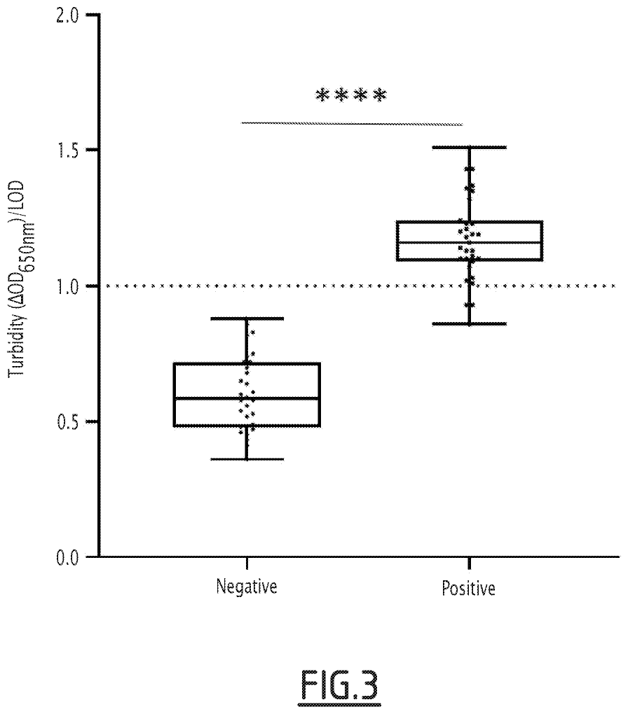

The results, as shown in Table 3 and FIG. 3, indicates that 28 out of the 31 DEN(+) clinical samples were identified by the RT-RPA-MFEA method. Two DENV4(+) samples out of six were not detected as positive after the MNP Agglutination assay, which may not be surprising as the primer pair used for RT-RPA, although enabling amplification of any serotypes, has more mismatch with DENV4 than with DENV1, DENV2 or DENV3.

Altogether, the assay designed for DEN virus detection has an accuracy of 94.64% (sensitivity of 90.32 and specificity of 100%) (see Table 4).

TABLE 3
Full data set of DENV (+) clinical
samples used in the molecular MFEA readout

| | | Real Time RT PCR | MNP Agglutination |
|---|---|---|---|
| Serotype | Sample | (Ct value) | Turbidity |
| DENV1 | 1 | 28 | + |
| | 2 | 9 | + |
| | 3 | 12 | + |
| | 4 | 8 | + |
| | 5 | 11 | + |
| | 6 | 19 | + |
| | 7 | 27 | + |
| | 8 | 14 | + |
| | 9 | 13 | + |
| | 10 | 9 | + |
| | 11 | 25 | + |
| | 12 | 28 | + |
| | 13 | 31 | + |
| | 14 | 13 | + |
| | 15 | 29 | + |
| | 16 | 19 | + |
| | 17 | 33 | − |
| | 18 | 18 | + |
| DENV2 | 19 | 10 | + |
| | 20 | 12 | + |
| | 21 | 16 | + |
| | 22 | 14 | + |
| | 23 | 14 | + |
| | 24 | 16 | + |
| DENV3 | 25 | 18 | + |
| DENV4 | 26 | 14 | + |
| | 27 | 19 | − |
| | 28 | 10 | + |
| | 29 | 27 | + |
| | 30 | 11 | + |
| | 31 | 13 | − |
| Total | 31 | / | 28 |

TABLE 4
Molecular MFEA readout on biological samples

| | | Samples | Diagnostic | Diagnostic | |
|---|---|---|---|---|---|
| Sample | Samples, | correctly | sensitivity, | specificity, | Accuracy, |
| type | n | detected, n | % (95% CI) | % (95% CI) | % |
| DENV | 31 | 28 | 90.32 | / | 94.64 |
| | | | (79.91-100) | | |
| Healthy | 30 | 30 | / | 100 | |

DENV, dengue virus;
CI, confidence interval
*[number of positive samples/(number of positive samples + number of false-negative samples)] × 100
† [number of negative samples/(number of negative samples + number of false-positive samples)] × 100
‡ [(number of negative samples + number of positive samples)/(number of negative samples
+ number of positive samples + number of false-negative samples + number of false-positive samples)] × 100

### SARS-COV2 RT-RPA Amplification

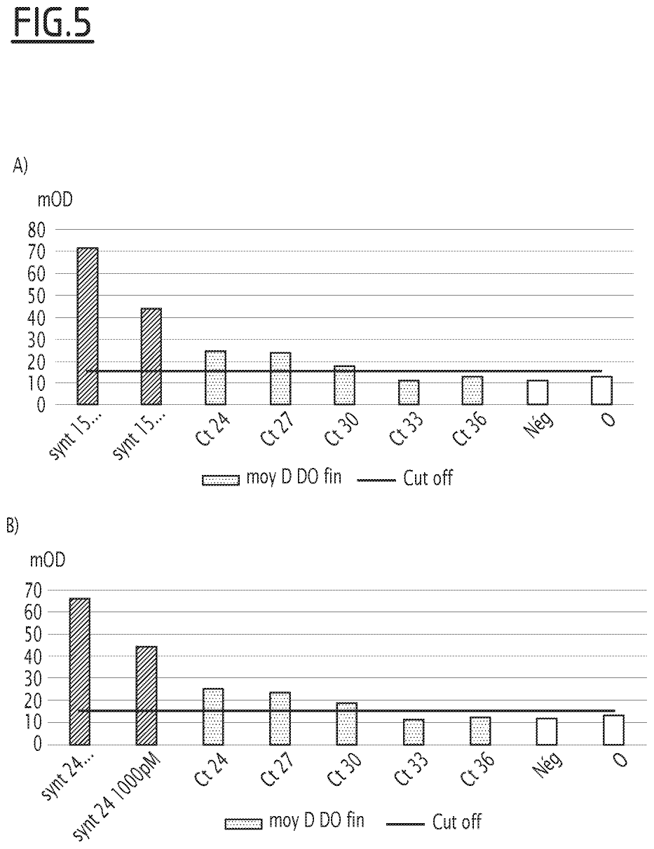

A series of SARS-COV2 RNA materiel was constituted ranging from Ct24 to Ct36 and assayed by RT-RPA-MEFA assay. The limit of detection of the method is evaluated between Ct30 and Ct33. No effect was observed in using a 24-mer probe rather than a 15-mer probe for the MNP agglutination assay (FIG. 5).

### DETAILED DESCRIPTION OF EMBODIMENTS

The method aims at the molecular detection of an infectious agent based on isothermal amplification of infectious agent's DNA or RNA by RPA or RT RPA respectively, combined with MFEA readout. The method enables for determining if a subject is infected with an infectious agent, or if the infectious agent is present in the environment.

### Infectious Agent

The infectious agent is a bacterium or a virus.

According to an embodiment, the infectious agent is a virus, such as a RNA virus (single stranded or double stranded) or a DNA virus (single stranded or double stranded).

According to certain embodiments, the virus is a RNA virus, e.g. a RNA virus of the Flaviviridae Family, Hepadnaviridae Family, Bunyaviridae Family, Filoviridae Family, Togaviridae Family, Coronaviridae Family, Rhabdoviridae Family or Retroviridae Family.

According to certain embodiments, the virus is a RNA virus of the Flaviviridae Family, such as a virus of the Flavivirus Genus, e.g. Dengue virus, Japanese encephalitis virus, Tick-borne encephalitis virus, West Nile virus, Usutu, Yellow fever virus, or Zika virus, or a virus of the Hepacivirus Genus such as hepatitis C virus, Pegivirus or Pestivirus Genus.

According to certain embodiments, the virus is a RNA virus of the Filoviridae Family, such as an Ebola virus.

According to certain embodiments, the virus is a virus of the Togaviridae Family, of the Alphavirus Genus, such as Chikungunya virus.

According to certain embodiments, the virus is a RNA virus of the Coronaviridae Family, in particular a virus of the species Severe acute respiratory syndrome-related coronavirus, more particularly SARS-CoV-2.

According to certain embodiments, the virus is a RNA virus of the Rhabdoviridae Family, especially of the Genus Lyssavirus, such as rabies virus.

According to certain embodiments, the virus is a RNA virus of the Retroviridae Family, especially human Immunodeficiency virus (HIV).

According to certain embodiments, the virus is a DNA virus of the Hepadnaviridae Family, especially hepatitis B virus (HBV).

According to an embodiment, the infectious agent is a bacterium. The bacterium is for instance a food poisoning bacterium (such as E. coli, Salmonella, or Shigella), a tuberculosis bacterium, the bacterium responsible for Lyme disease (Borrelia burgdorferi. B. burgdorferi), Vibrio cholerae, Vibrio cholerae, Bordetella pertussis, or a bacterium responsible for urinary tract infection (UTI) such as Escherichia coli, Klebsiella pneumoniae, Proteus mirabilis, Pseudomonas aeruginosa, or Enterococcus faecalis.

According to the method of detection, a single or multiplexed assay may be conducted to detect simultaneously one or more infectious agents.

### Sample Likely to Contain the Infectious Agent and Nucleic Acid Extract

The sample likely to contain the infectious agent is a biological sample, or an environmental sample.

The biological sample likely to contain the infectious agent is usually a blood sample, a plasma sample, a serum sample, a saliva sample, a urine sample, a nasopharyngeal swab, a vaginal swab, sputum, cerebrospinal fluid, or a dry blood spot. The biological sample is taken from a human subject, or from a non-human animal.

The environmental sample is usually a wastewater sample, a food sample, or a plant sample.

A nucleic acid extract can be readily isolated from the sample by methods known to the skilled in the art. For instance, a blood sample would be treated by lysing blood cells and purifying nucleic acids from the lysate, for instance using a commercial kit, such as MagNA Pure Compact Nucleic Acid Isolation Kit I (Roche Diagnostics, Mannheim, Germany).

The nucleic acid extract used in the frame of the method of detection is preferably provided in a volume ranging from 0.1 to 200 μL, such as 2-100 μL, 2-20 μL, 5-15 μL, or 5-10 μL.

### Recombinase Polymerase Amplification (RPA)

Recombinase polymerase amplification is an in vitro method for the exponential amplification of target nucleic acids wherein high recombinase activity is maintained in a highly dynamic recombination environment, supported by ATP.

RPA involves cyclic repetition of three steps: first, a recombinase agent is contacted with a first and a second nucleic acid primer to form a first and a second nucleoprotein primer. Second, the first and second nucleoprotein primers are contacted to a double stranded target sequence to form a first double stranded structure at a first portion of said first strand and form a double stranded structure at a second portion of said second strand so the 3′ ends of said first nucleic acid primer and said second nucleic acid primer are oriented towards each other on a given template DNA molecule. Third, the 3′ end of said first and second nucleoprotein primers are extended by DNA polymerases to generate first and second double stranded nucleic acids, and first and second displaced strands of nucleic acid. Finally, the second and third steps are repeated until a desired degree of amplification is reached (see e.g. US 20030219792). The RPA reaction is usually conducted at 37-42° C.

To amplify (and then detect) simultaneously one or more infectious agents, multiplex RPA reactions are implemented and multiplex RPA reaction products are detected, for instance as described in patent application US20200095584.

The target sequence(s) to be amplified, is(are) preferably a double stranded DNA. However, the RPA amplification is not limited to double stranded DNA because other nucleic acid molecules, such as a single stranded DNA or RNA can be turned into double stranded DNA by one of skill in the arts using known methods. Suitable double stranded target DNA may be a genomic DNA or cDNA of the infectious agent(s).

Typically, when the infectious agent's genomic nucleic acid is RNA, the nucleic acid extract is submitted to reverse transcription, to reverse transcribe infectious agent's RNA into DNA. This can be readily achieved just by adding a reverse transcriptase, preferably a reverse transcriptase functional at 37-42° C., to the RPA reaction mix when setting up a RPA reaction. The RNA is then reverse transcribed and the DNA produced and amplified all in one step.

The nucleic acid extract, optionally submitted to reverse transcription, is submitted to recombinase polymerase amplification, using a pair of primers targeting a region of the infectious agent's DNA, wherein one primer of the primer pair is bound to a first member of a binding pair. The recombinase polymerase amplification generates double stranded DNAs (amplicons), wherein one strand of the double stranded DNAs is bound to the first member the binding pair, e.g. to biotin.

As used herein, a “binding pair” denotes a molecular pair, in particular a protein pair, usually having high affinity (e.g subnanomolar), which is conventionally used in bioassay. A binding pair typically includes ligand:receptor couples such as avidin:biotin or barstar:barnase, or any antigenic tag: anti-tag antibody. According to an embodiment, the binding pair comprise (i) biotin and (ii) avidin, or an avidin derivative (e.g. streptavidin, or neutravidin), or an anti-biotin antibody, preferably an anti-biotin monoclonal antibody. Preferably, the first member of the binding pair is biotin

For instance, the forward primer targeting a region of the infectious agent's DNA is bound at its 5′end to said first member of the binding pair, e.g. biotin.

The second member of the binding pair is introduced in the reaction mix after the RPA reaction is completed, during the magnetic-field enhanced agglutination assay.

The primers targeting a region of the infectious agent's DNA are typically 15-45 nucleotide long, preferably 30-40 nucleotide long as RPA preferably uses longer primer sequences than PCR.

When the infectious agent exists as several serotypes (for instance there are 4 serotypes of dengue viruses, 47 serotypes of Shigella divided into 4 groups), the primer pair is designed either to detect specifically a given serotype of the infectious agent (‘specific’ primers), or to detect all serotypes or a subgroup of the serotypes of the infectious agent (‘consensus degenerate’ primers).

Typically, to amplify dengue viruses DNA, the primer pair may target the DNA of the DEN 3′NTR gene, using for instance a forward primer comprising or consisting of SEQ ID NO: 1, and a reverse primer comprising or consisting of SEQ ID NO: 2.

Typically, to amplify SARS-COV2 DNA, the primer pair may target the DNA of the SARS-COV2 S gene, using for instance a forward primer comprising or consisting of SEQ ID NO: 3, and a reverse primer comprising or consisting of SEQ ID NO: 4.

The recombinase polymerase amplification therefore produces a RPA reaction product comprising amplicons which are double stranded DNAs, wherein one of the strands of the amplicons is bound to the first member of the binding pair, e.g. to biotin.

The recombinase polymerase amplification, including reverse transcription where necessary, typically lasts 25-40 min.

The recombinase polymerase amplification is preferably followed by a step of denaturation of the double stranded DNAs (amplicons) obtained after recombinase polymerase amplification to obtain single stranded DNAs, wherein a part of the single stranded DNAs is bound to the first member the binding pair, e.g. to biotin. The denaturation is either performed by thermal denaturation, chemical denaturation such NAOH denaturation, or enzymatic denaturation of the RPA reaction product. Thermal denaturation can be implemented e.g. by incubating the RPA reaction product at 90-100° C., such as about 95° C., during 8-12 min, preferably about 10 min, followed by cooling on ice for 2-5 min. Chemical denaturation can be implemented e.g. by alcalinization and neutralization of the RPA reaction product; for instance by incubating the RPA reaction product with NaOH, e.g. NaOH 0.3-0.5 N, at room temperature for 3-10 min, e.g. 4-6 min, followed by neutralization, by addition of the same normality of a strong acid such as acetic acid. Enzymatic denaturation can be implemented using a T7 exonuclease, for instance.

Altogether, performing the recombinase polymerase amplification, including reverse transcription where necessary, and denaturation takes about 30-45 min.

### Magnetic-Field Enhanced Agglutination Assay

The design of a DNA agglutination assay can be as simple as biotinylated double stranded DNA targets incubated with streptavidin-linked magnetic beads. However, many situations require target DNA sequences to be discriminated from non-specific amplification or DNA sequences of high homology, such as identifying a viral strain among a family of viruses. To gain in specificity, a probe is added to the assay, modifying the design of the chaining process with a third component, the probe-linked magnetic beads.

The principle of MFEA was described in the international patent applications WO 03/044532 and WO 2014/140468, and published in Daynes et al. Chem. 2015, 87, 15, 7583-7587. The technique is based on the acceleration of the recognition rate between members of a binding pair, e.g. ligands and receptors, induced by magnetic forces.

MFEA uses two sets of magnetic beads: (i) a first set of magnetic beads coated with a nucleic acid probe having complementarity with the strand of the DNA amplicons produced by the recombinase polymerase amplification that is bound to the first member the binding pair (e.g. biotin), and (ii) a second set of magnetic beads coated with the second member of the binding pair (e.g. avidin, or an avidin derivative (e.g. streptavidin, or neutravidin), or an anti-biotin antibody). MFEA thus comprises contacting the single stranded DNAs bound to the first member of the binding pair (obtained after recombinase polymerase amplification as part of the double stranded DNA amplicons, or after recombinase polymerase amplification and denaturation as single stranded DNAs bound to the first member of the binding pair) with (i) a first set of magnetic beads coated with a nucleic acid probe having complementarity with the single stranded DNAs bound to the first member of the binding pair, and (ii) a second set of magnetic beads coated with the second member of the binding pair.

The inventors have shown that the DNA of the infectious agent amplified by recombinase polymerase amplification can be detected by magnetic-field enhanced agglutination without interference of the components, in particular the enzymes (recombinase, polymerase) of the reaction mix used for recombinase polymerase amplification.

The magnetic beads are magnetic microparticles or magnetic nanoparticles (MNPs), preferably MNPs. The magnetic beads are paramagnetic, diamagnetic, ferromagnetic or ferrimagnetic or else superparamagnetic micro- or nano-particles.

The nucleic acid probe is typically DNA, and 10-40, or preferably 15-30 nucleotide long. The nucleic acid probe is fully or partially complementary over its entire sequence with the strand of the DNA amplicons produced by the recombinase polymerase amplification that is bound to the first member the binding pair.

For instance, a nucleic acid probe comprising or consisting of SEQ ID NO:5 can be used to detect dengue virus amplicons produced by RPA using the primer pair comprising or consisting of SEQ ID NO: 1 (forward) and SEQ ID NO: 2 (reverse).

For instance also, a nucleic acid probe comprising or consisting of SEQ ID NO:6 or SEQ ID NO: 7 can be used to detect SARS-COV2 virus amplicons produced by RPA using the primer pair comprising or consisting of SEQ ID NO: 3 (forward) and SEQ ID NO: 4 (reverse).

According to an embodiment, to gain in signal intensity, nucleic acid probes are grafted onto the first set of magnetic beads through a polythiolated link, preferably a tetrathiolated link, at their 5′ end, as described in the international patent applications WO2013150106 and WO2013150122. Tetrathiolated probes have been previously evaluated in a microplate format and performed better in detecting viral genomes than ester link probes (Lereau et al., Anal. Chem. 85 (19) (2013) 9204-9212; Armbruster et al., Clin. Biochem. Rev. 29 (Suppl 1) (2008) S49-S52).

The 5′-polythiolated nucleic acid probes have the following formula:

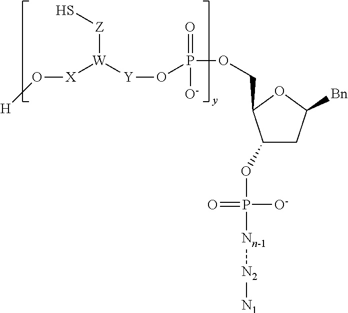

in which,
   n is an integer comprised between 1 and 14,
   y is an integer comprised between 1 and 12, preferably 4,
   N₁, . . . Nₙ₋₁ represent, independently of one another, a nucleotide of the nucleic acid probe,
   W is selected from C1-C6 alkane triyl groups, C6-C12 aryl triyl groups and C6-C12 aralkane triyl groups, wherein the C1-C6 alkane triyl group is a linear or branched C1-C6 alkane triyl substituted by at least two alkyl groups,
   Z is selected from C1-C6 alkoxy groups, oxygen-containing or nitrogen-containing C3-C6 cycloheteroalkyl groups, C1-C6 NCO-alkyl groups, C1-C6 CON-alkyl groups,
   Y is selected from linear or branched C1-C6 alkyl groups, C1-C6 aminoalkyl groups, C1-C6 alkoxy groups, C3-C6 cycloalkyl groups, oxygen-containing or nitrogen-containing C3-C6 cycloheteroalkyl groups,
   X is selected from linear or branched C1-C6 alkyl groups, C1-C6 aminoalkyl groups, C1-C6 alkoxy groups, C3-C6 cycloalkyl groups, oxygen-containing or nitrogen-containing C3-C6 cycloheteroalkyl groups, and
   Bn represents the nucleobase of the n^{th} nucleotide (at the 5′ end of the nucleic acid probe).

Preferably, W is a C1-C6 alkane triyl group, preferably —(CH₃)—.

Preferably, Z is a C1-C6 alkoxy group.

Preferably, X and Y are each a linear C1-C6 alkyl group.

The nucleobase Bn is a purine base, pyrimidine base, or a derivative thereof (i.e. a modified nucleobase).

The 5′-polythiolated nucleic acid probes are grafted on a substrate, covering partially or totally the magnetic beads.

According to an embodiment, the substrate is a film of gold or platinum, preferably of gold.

In another embodiment, the substrate is a polymer, for example polystyrene, which is grafted with alkenyl or alkynyl or bromoacetamides or iodoacetamides functions. According to an embodiment, the alkenyl or alkynyl functions are activated by a carbonyl function in alpha position; preferably the alkenyl or alkynyl or bromoacetamides or iodoacetamides functions are chosen from maleimide, acrylamide, iodoacetamido or bromoacetamido, 2-propynamide or N-alkyl-2-propynamide groups.

For example, the magnetic beads may comprise receiving zones covered with a film of gold or platinum or covered with alkenyl or alkynyl functions, such as acrylamide or maleimide functions on which 5′-polythiolated nucleic acid probes are deposited.

As described in the international patent applications WO2013150106 and WO2013150122, the thiol function of the 5′-polythiolated nucleic acid probes reacts with the carbon-carbon double bond or triple bond carbon-carbon activated by a carbonyl function in alpha position.

According to an embodiment, the magnetic beads are covered with maleimide or acrylamide groups, and the surface of the magnetic bead is functionalized with the nucleic acid probes by creating thioether bond(s).

According to an embodiment, the magnetic beads are covered with a gold surface and the surface of the magnetic bead is functionalized with the nucleic acid probes by creating gold-sulphur bond(s).

The attachment of 5′-polythiolated nucleic acid probes to the surface of the magnetic beads occurs by contact of the surface magnetic beads to be treated with a solution comprising the 5′-polythiolated nucleic acid probes. Generally, one or more subsequent washing and drying steps are provided. In general, the 5′-polythiolated nucleic acid probes solution is at a concentration comprised between 0.10 μM and 500 μM, preferably between 0.50 μM and 100 μM for the gold surface and between 50 and 200 nM, preferably between 75 nM and 150 nM for the maleimide or acrylamide, followed by washing to remove the unreacted products.

According to an embodiment, the magnetic beads are monodispersed and super-paramagnetic beads composed of magnetic core encapsulated by a highly cross-linked hydrophilic polymer shell (e.g. Carboxyl-Adembeads from Ademtech, 200 nm magnetic nanoparticles). The surface is activated with carboxylic acid functionality. The 5′-polythiolated, preferably 5′tetrathiolated, nucleic acid probes are covalently grafted on the magnetic beads as follows: (A) the magnetic beads are incubated with 1-ethyl-3-[3-(dimethylamino)propyl] carbodiimide hydrochloride to form an ester active intermediate, then with amino-PolyEthyleneGlycol(PEG)-maleimide; (B) The 5′-polythiolated, preferably 5′-tetrathiolated, nucleic acid probes are incubated with tris(2-carboxyethyl)phosphine hydrochloride in presence of Na₂HPO₄, NaCl, and EDTA to reduce the disulfide bonds; (C) the PEG-maleimide magnetic beads of (A) are then incubated with the 5′-polythiolated, preferably 5′-tetrathiolated, nucleic acid probes of (B); (D) the magnetic beads are the blocked by incubating with Tris-HCL and cysteine, and washed. A detailed protocol is described in Example 1.

The presence of several sulfur atoms on the 5′-polythiolated nucleic acid probes allows creating several gold-sulfur bonds, or several thioether bonds, which can stabilize the 5′-nucleic acid probes on the surface of the magnetic beads.

In the second set of magnetic beads, the beads are covered partly or totally with the second member of the binding pair, e.g. avidin, or an avidin derivative (e.g. streptavidin, or neutravidin), or an anti-biotin antibody if the first member of the biding pair is biotin.

For conducting the MFEA assay, the single stranded DNAs bound to the first member the binding pair are contacted with (i) the first set of magnetic beads coated with the nucleic acid probe having complementarity with the single stranded DNAs bound to the first member the binding pair, and (ii) the second set of magnetic beads coated with the second member of the binding pair, and then submitted to magnetic-field enhanced agglutination.

The contacting can be simply implemented by adding the first and second sets of magnetic beads into the denatured RPA reaction product.

Magnetic-field enhanced agglutination is performed by submitting the mixture containing the first and second sets of magnetic beads and denatured RPA reaction product to one or more cycles of magnetization and relaxation. Magnetic-field enhanced agglutination typically comprises or consists of 1-10, 2-5 or 3-4 cycles of magnetization and relaxation. The magnetization typically comprise or consists in applying a magnetic field of 3-100 mT, 5-30 mT, preferably 10-20 mT, e.g. 13-17 mT for a duration of 1 to 300 s, preferentially of 20 to 120 s, advantageously 30-80 s, e.g. about 50-70 s. Magnetization alternates with periods of relaxation where no magnetic field is applied. Relaxation periods typically lasts 1 to 300 s, preferentially of 10 to 120 s, advantageously 20-40 s, e.g. 25-35 s. According to an embodiment, magnetic-field enhanced agglutination comprises or consists of 2-4 cycles, preferably 3 cycles, of magnetization (13-17 mT, preferably about 15 mT, for 50-70 s, preferably about 60 s) and relaxation (for 20-40 s, preferably about 30 s).

The agglutination state measured before and after magnetic-field enhanced agglutination is compared with a control to determine if the sample is positive for the infectious agent.

According to an embodiment the agglutination state is assayed by measuring turbidity. According to this embodiment, turbidity of the mixture of the denatured RPA reaction product and first and second sets of magnetic beads is measured before and after having conducted the magnetic-field enhanced agglutination, at the end of the cycles of magnetization and relaxation. Turbidity of the mixture is evaluated by measuring optical density, for instance optical density at 650 nm (A OD650 nm).

The method then comprises comparing variation of the agglutination state, n particular variation of turbidity, measured before and after magnetic-field enhanced agglutination with a control to determine if the sample is positive for the infectious agent. The control value can be a cut-off value, determined beforehand typically by implementing the method on samples containing serial dilutions of the infectious agent's nucleic acids, and on one or more control samples (blank and/or negative sample). The control value can be the variation of agglutination state, in particular of turbidity, in a control sample (blank and/or negative sample) submitted in parallel to the (RT)-RPA-MFEA method. A variation of the agglutination state, in particular of turbidity, above the control indicates that the infectious agent was present in the sample (sample is positive for the infectious agent), while a variation of the agglutination state, in particular of turbidity, equal or below the control indicates that the infectious agent was not present, or not present in detectable amount, in the sample (sample is negative for the infectious agent).

According to an embodiment, the sample likely to contain the infectious agent is an environmental sample, and the method further comprises implementing an immune assay on the environmental sample (i.e. a fraction of the same sample from which the nucleic acid extract was prepared, or another sample of the same source). The immune assay aims at detecting the infectious agent, by detecting e.g. a protein, in particular an antigen, of the infectious agent in the sample.

Method for Determining if a Subject is or has been Infected with an Infectious Agent

The method for detecting an infectious agent, which comprises submitting a nucleic acid extract of a biological sample likely to contain the infectious agent to (RT)-RPA-MFEA enables for determining if a subject is (or is or has been) infected with an infectious agent.

Accordingly, the method enables for determining if a subject is infected with an infectious agent, which comprises implementing a method for detecting an infectious agent as described above on a biological sample of the subject likely to contain nucleic acids of the infectious agent; and determining that the subject is infected if the infectious agent is present in the biological sample.

In particular the method may further comprise implementing an immune assay comprising, or consisting of, a serological assay to determine if the subject has antibodies directed against the infectious agent and/or antigens of the infectious agent, and/or a cellular assay on biological sample to determine if a cell is activated upon infection; and determining that the subject is or has been infected based on the result of the method for detecting the infectious agent and/or the immune assay.

The biological sample for the serological assay is a biological sample of the patient containing serum, typically whole blood, plasma, serum, a vaginal swab, sputum, cerebrospinal fluid, or whole blood spot.

Advantageously, the serological and/or immune assay is a magnetic-field enhanced agglutination assay using magnetic beads coated with an antigen of the infectious agent or an antibody thereto.

1. An in vitro method for detecting an infectious agent, comprising:
submitting a nucleic acid extract of a sample likely to contain the infectious agent to a recombinase polymerase amplification, followed by magnetic-field enhanced agglutination assay; and
determining if the infectious agent is present in the sample based on the result of the magnetic-field enhanced agglutination assay.

2. The in vitro method of claim 1, further comprising:
providing a nucleic acid extract of the sample likely to contain the infectious agent;
if the infectious agent's genomic nucleic acid is RNA, submitting the nucleic acid extract to reverse transcription, to reverse transcribe infectious agent's RNA into DNA;
submitting the nucleic acid extract to recombinase polymerase amplification, using a pair of primers targeting a region of the infectious agent's DNA, wherein one primer of the primer pair is bound to a first member of a binding pair;
optionally, denaturing double stranded DNAs obtained after recombinase polymerase amplification to obtain single stranded DNAs, wherein a part of the single stranded DNAs is bound to the first member of the binding pair;
contacting the single stranded DNAs bound to the first member of the binding pair with (i) a first set of magnetic beads coated with a nucleic acid probe having complementarity with the single stranded DNAs bound to the first member of the binding pair, and (ii) a second set of magnetic beads coated with the second member of the binding pair;
submitting the single stranded DNAs bound to the first member of the binding pair, and first and second sets of magnetic beads to magnetic-field enhanced agglutination; and
comparing variation of agglutination state measured before and after magnetic-field enhanced agglutination with a control to determine if the sample is positive for the infectious agent.

3. The in vitro method of claim 1, wherein the sample likely to contain the infectious agent is a biological sample or an environmental sample.

4. The in vitro method of claim 1, wherein the infectious agent is a bacterium or a virus.

5. The in vitro method of claim 1, wherein the infectious agent is a RNA virus.

6. The in vitro method of claim 1, wherein the infectious agent is a RNA virus of the Flaviviridae Family, Hepadnaviridae Family, Bunyaviridae Family Filoviridae Family, Toagviridae Family, Coronaviridae Family, or Rhabdoviridae Family.

7. The in vitro method of claim 1, wherein the nucleic acid probe having complementarity with the single stranded DNAs bound to the first member of the binding pair is 5′-polythiolated and is covalently grafted to the first set of magnetic beads.

8. The in vitro method of claim 1, wherein the second set of magnetic beads are covered partly or totally with the second member of the binding pair.

9. The in vitro method of claim 1, wherein the magnetic beads of the first and second sets of magnetic beads are magnetic micro- or nano-particles.

10. The in vitro method of claim 1, wherein the magnetic-field enhanced agglutination comprises 1-10 cycles of magnetization and relaxation.

11. The in vitro method of claim 10, wherein the magnetization comprises applying a magnetic field of 3-100 mT, for a duration of 1 to 300 s, and relaxation lasts 1 to 300 s.

12. The in vitro method of claim 10, wherein the magnetic-field enhanced agglutination comprises 2-4 cycles of magnetization at 13-17 mT, for 50-70 s, and relaxation for 20-40 s.

13. The in vitro method of claim 1, wherein the first member of the binding pair is biotin, and the second member of the binding pair is avidin, or an avidin derivative, or an anti-biotin antibody.

14. The in vitro method of claim 1, wherein the sample likely to contain the infectious agent is an environmental sample, and the method further comprises implementing an immune assay on the environmental sample.

15. An in vitro method for determining if a subject is infected with an infectious agent, comprising:
implementing a method for detecting an infectious agent according to claim 1 on a biological sample of the subject likely to contain nucleic acids of the infectious agent; and
determining that the subject is infected if the infectious agent is present in the biological sample.

16. An in vitro method for determining if a subject is or has been infected with an infectious agent, comprising:
implementing a method for detecting the infectious agent according to claim 1 on a nucleic acid extract of a biological sample of the subject likely to contain the infectious agent;
implementing an immune assay comprising a serological assay to determine if the subject has antibodies directed against the infectious agent and/or antigens of the infectious agent, and/or a cellular assay on biological sample to determine if a cell is activated upon infection; and
determining that the subject is or has been infected based on the result of the method for detecting the infectious agent and/or the immune assay.
